(19) United States
(12) Patent Application Publication
(10) Pub. No.: US 2014/0066710 A1
(43) Pub. Date: Mar. 6, 2014

(54) DEVICES AND METHODS FOR INTRAOPERATIVE CONTROL OF ENDOSCOPIC IMAGING

(71) Applicant: University of Iowa Research Foundation, Iowa City, IA (US)

(72) Inventors: Christopher Graves, Iowa City, IA (US); Brian Wolf, Iowa City, IA (US)

(21) Appl. No.: 13/921,691

(22) Filed: Jun. 19, 2013

Related U.S. Application Data
(60) Provisional application No. 61/661,657, filed on Jun. 19, 2012.

(51) Int. Cl. A61B 1/00; A61B 1/317; A61B 1/04; A61B 5/06; A61B 1/07

(52) U.S. Cl. CPC A61B 1/00006; A61B 5/065; A61B 1/0016; A61B 1/00163; A61B 1/00039; A61B 1/00066; A61B 1/07; A61B 1/042; A61B 1/317; A61B 1/00071
USPC 600/109; 600/117

(57) ABSTRACT

An endoscopic imaging system including a rigid endoscope, a housing, a user input assembly, control circuitry, an actuator, and a position sensor. The endoscope defines a centrifugal bore and has a longitudinal axis, a proximal end portion, and a distal end portion. The endoscope includes a lens assembly positioned within the centrifugal bore. The housing of the device has an internal compartment and is securely coupled to the proximal end portion of the endoscope. Optical coupling for light is provided which allows for unlimited rotation and provides for improved image stability. The user input assembly receives user input controls corresponding to a selected rotational position of the endoscope. The control circuitry receives the user input controls and transmits them to the actuator, which controllably rotates the endoscope about the longitudinal axis of the endoscope in response to the user input controls. The position sensor produces a position signal indicative of the rotational position of the endoscope. The control circuitry uses the position signal to determine the required axial rotation of the endoscope.

### CLAIM OF PRIORITY

This application claims priority from U.S. Provisional Patent Application No. 61/661,657 filed on Jun. 19, 2012, which is relied upon and incorporated herein in its entirety by reference.

### FIELD

This invention relates to devices and methods for controlling endoscopic imaging. More particularly, this invention relates to devices and methods for selectively controlling the rotation of an endoscope such that a desired field of view is achieved.

### BACKGROUND

An endoscope is a surgical instrument used to visualize the interior of a hollow viscus or cavity of a patient, such as the interior of a joint (arthroscope). Endoscopes typically include: a rigid or flexible tube, a light delivery system and a lens system that transmits an image from an objective lens to a user. A camera is often coupled to the endoscope, and optical focusing of the camera is usually performed by a rotating a manual ring at the camera's base. The light delivery system typically includes an optical fiber system for directing light from outside the patient to the end of the endoscope positioned within the patient. In a conventional setup, a fiber optic cable from an external light source is connected to the endoscope in a plane orthogonal to the line defined by the long axis of the endoscope.

Conventional procedures that require the use of rigid endoscopes are performed using two hands. In these procedures, as shown in FIG. 10, the surgeon uses one hand to hold the rigid endoscope (usually with attached video camera) and a second hand to hold a surgical tool, such as a blunt probe, spinning burr, or shaver. However, as shown in FIG. 9, the conventionally configured rigid endoscope itself was designed to be a two-handed tool, with the surgeon holding the camera portion with one hand and using the other hand to manipulate the angle at which the light post (and field of view) rotates, and adjust the focus if necessary. Thus, it is difficult for the surgeon to operate both the rigid endoscope and the surgical instruments to be used in conjunction with the endoscope.

These difficulties are typically overcome through the use of an assistant, or attempting a non-ergonomic stretch of one hand such that the light post, and thus the field of view of the endoscope is adjusted with the surgeon's small finger while the remaining fingers are used to stabilize the rigid endoscope and the camera. These clinical workarounds are clumsy. Additionally, rotation of the handle provides both (a) torque in the axial direction of the scope and (b) a force normal to the vector direction of the endoscope. This causes the camera angle to tend to skew off the desired viewing direction if it is not in a firm control and also causes a jitter in the arthroscope image. The clinical alternative is simply tolerating a sub-optimal field of view until the surgeon has an opportunity to remove the other surgical instruments from the surgical field and devote both hands to control of the endoscope.

A rotational viewing limitation is also inherent in the method of the construction of the conventional endoscope. The light post can be rotated by less than 360 degrees. This creates a surgical blind spot. With a conventional endoscope, if the surgeon wants to see just beyond the rotational limitation, the scope must be rotated 360 degrees in the opposite direction.

Consequently, current methods do not permit a surgeon to ergonomically control the field of view of a rigid endoscope using one hand while maintaining a stable position of the endoscope and the camera.

Accordingly, there is a need in the pertinent art for devices and systems for one-handed control of the rotational field of view of a rigid endoscope in an ergonomic manner while maintaining stability of the endoscope and permitting control of secondary surgical instruments by the surgeon.

### SUMMARY

Described herein is an endoscopic imaging system. The endoscopic imaging system can include a rigid endoscope, a housing, a user input assembly, control circuitry, an actuator, and a position sensor. The endoscope defines a centrifugal bore and has a longitudinal axis, a proximal end portion, and a distal end portion. The endoscope includes a lens assembly positioned within the centrifugal bore. The lens assembly includes an objective lens positioned near the distal end portion of the endoscope and angled relative to the longitudinal axis of the endoscope. The housing of the device has an internal compartment and is securely coupled to the proximal end portion of the endoscope. The user input assembly can be configured to permit entry of user input controls corresponding to a selected rotational position of the endoscope. The control circuitry can be positioned within the internal compartment of the housing and can be positioned in operative communication with the user input assembly for purposes of receiving the user input controls. The actuator can be positioned within the internal compartment of the housing and in operative communication with the control circuitry. The actuator is configured to controllably rotate the endoscope about the longitudinal axis of the endoscope in response to the received user input controls. The position sensor can be positioned within the internal compartment of the housing and in operative communication with the control circuitry. The position sensor is configured to produce a position signal indicative of the rotational position of the endoscope. The control circuitry can include a processor that is configured to compare the rotational position of the endoscope with the selected rotational position. In use, the processor can determine a required axial rotation of the endoscope that is sufficient to position the endoscope in the selected rotation and can then instruct the actuator to effect the required axial rotation of the endoscope.

Optionally, the proximal end portion of the endoscope can define a coupling for a fiber optic light channel (referred to interchangeably herein as a “light guide”) circumferentially surrounding the longitudinal axis of the endoscope. The proximal end portion of the endoscope can also define a coupling for a fiber optic video channel (referred to herein as the “video channel”). Together, the light guide and the video channel comprise a transmission cable.

The endoscopic imaging system can further include a light source. The light guide can be operatively coupled to, and extend between, the light source and the aperture of the proximal end portion of the endoscope. The light guide can be configured to transmit light into the centrifugal bore of the endoscope substantially surrounding the longitudinal axis of the endoscope. Optionally, the user input assembly can permit entry of light activation controls that are transmitted by the control circuitry to the light source to thereby effect selective activation and inactivation of the light source.

The endoscopic imaging system can still further include a camera operatively coupled to the centrifugal bore of the endoscope for purposes of receiving an image produced by the lens assembly. Optionally, the transmission cable of the endoscopic imaging system can include a fiber optic channel and a video channel, and the fiber optic channel of the transmission cable can provide operative communication between the light source and the centrifugal bore of the endoscope, while the video channel can provide operative communication between the camera and the centrifugal bore of the endoscope.

### BRIEF DESCRIPTION OF THE FIGURES

These and other features of the preferred embodiments of the invention will become more apparent in the detailed description in which reference is made to the appended drawings wherein:

FIG. 1 is a partially transparent side perspective view of an exemplary device for controlling endoscopic imaging, as described herein.

FIGS. 2-5 are schematic depictions of exemplary input assemblies positioned thereon the control surface of the device of FIG. 1.

FIG. 6 is a schematic diagram showing the operative communication among the control surface, the microcontroller, the servo motor, the endoscope, and the position sensor of the device of FIG. 1.

FIG. 7 is an image showing a perspective view of an exemplary device for controlling endoscopic imaging, as described herein.

FIG. 8 is a schematic diagram of another exemplary device for controlling endoscopic imaging having a non-mechanical light source coupling as described herein.

FIGS. 9 and 10 display conventional arthroscopes as are known in the art.

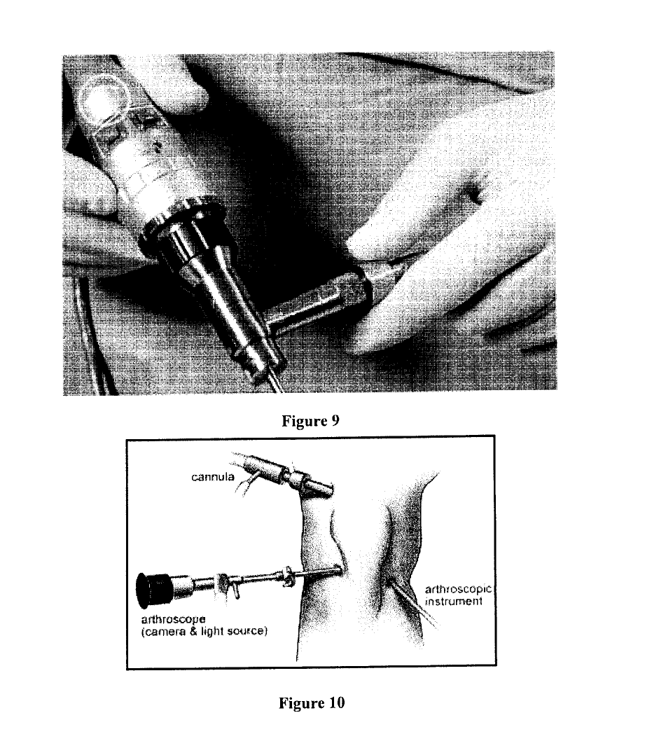

### DETAILED DESCRIPTION

The present invention can be understood more readily by reference to the following detailed description, examples, drawings, and claims, and their previous and following description. However, before the present devices, systems, and/or methods are disclosed and described, it is to be understood that this invention is not limited to the specific devices, systems, and/or methods disclosed unless otherwise specified, and, as such, can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used throughout, the singular forms “a,” “an” and “the” include plural referents unless the context clearly dictates otherwise. Thus, for example, reference to “a delivery conduit” can include two or more such delivery conduits unless the context indicates otherwise.

As used herein, the terms “optional” or “optionally” mean that the subsequently described event or circumstance may or may not occur, and that the description includes instances where said event or circumstance occurs and instances where it does not.

It is contemplated that the disclosed devices and systems can comprise elements of the devices and systems described in U.S. Pat. Nos. 5,184,602, 5,368,014, 6,364,830, 6,638,216, 6,929,603, and 7,175,593 and U.S. Patent Publication No. 2011/0201886, the disclosures of which are incorporated herein by reference in their entireties.

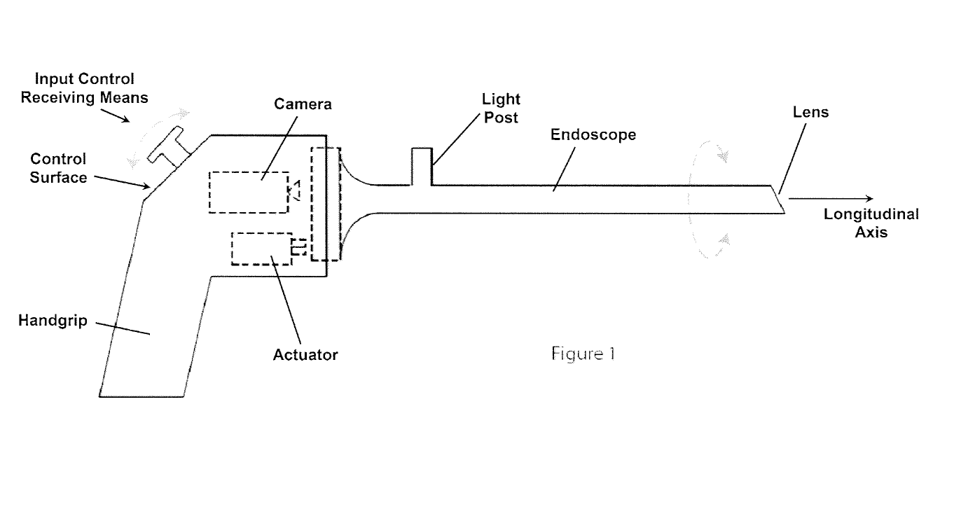

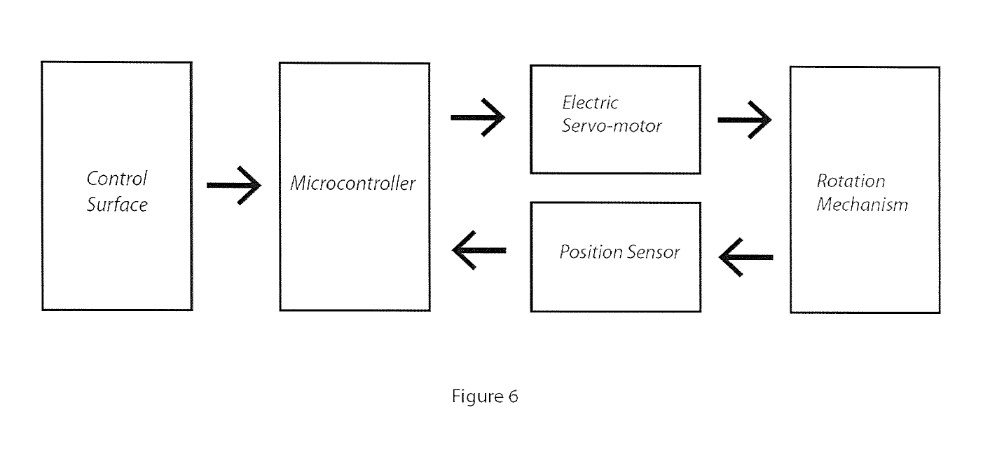

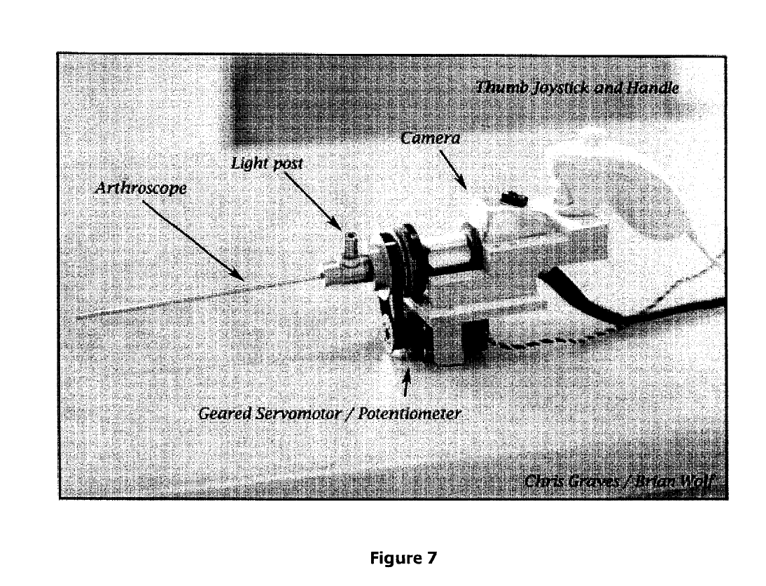

In one embodiment, and with reference to FIGS. 1-8, the invention relates to an endoscopic imaging system. In one aspect, the system can comprise a rigid endoscope defining a centrifugal bore and having a longitudinal axis, a proximal end portion, and a distal end portion. In this aspect, it is contemplated that the centrifugal bore can be substantially centrally positioned within the endoscope along the longitudinal axis of the endoscope. In another aspect, the endoscope can comprise a lens assembly positioned therein the centrifugal bore. In this aspect, the lens assembly can comprise an objective lens positioned proximate the distal end portion of the endoscope. It is contemplated that the lens assembly can further comprise conventional means for transmitting an image from the objective lens to a viewer, including, for example and without limitation, a relay lens system, a fiber optic bundle, and the like. In a further aspect, the objective lens can be angularly oriented relative to the longitudinal axis of the endoscope. For example, it is contemplated that the objective lens can be angularly oriented relative to the longitudinal axis of the endoscope by about 30° to about 70°. However, it is contemplated that the objective lens can be positioned at any angular orientation relative to the longitudinal axis of the endoscope, provided the lens assembly is capable of transmitting a useful image of a selected tissue region. Due to the angular orientation of the objective lens, it is understood that rotation of the endoscope produces a corresponding rotation of the objective lens, thereby adjusting the image produced by the lens assembly. As discussed herein, the rotation of the endoscope is done for purposes of adjusting the orientation of the objective lens, and thus, the particular image that is produced by the lens assembly.

Optionally, the proximal end portion of the endoscope can cooperate with the centrifugal bore to define a conventional viewing portal along the longitudinal axis of the endoscope. In this conventional configuration, the viewing portal is surrounded by a fiber optic channel coupled to a light post and permitting transmission of light. Alternatively, in another optional aspect, the proximal end portion of the endoscope can define an aperture that circumferentially surrounds the longitudinal axis of the endoscope and is optically coupled to the proximal end portion of the endoscope in a plane orthogonal to the longitudinal axis of the endoscope, thereby providing a coupling for a light guide as described herein.

In another aspect, the system can comprise a housing having an internal compartment and an outer surface. In this aspect, the housing can be securely coupled to the proximal end portion of the endoscope. In exemplary aspects, the housing can define a distal opening that is configured to complementarily receive the proximal end portion of the endoscope. In various aspects, a portion of the outer surface of the housing can define a control surface. In a further aspect, the housing can comprise a handgrip portion. In this aspect, the handgrip portion of the housing can be configured to conform to the shape of a user's hand and extend outwardly from the remainder of the housing such that the handgrip portion can be easily received within a user's hand. It is contemplated that the control surface of the housing can be positioned proximate the handgrip portion of the housing such that a user can simultaneously grip the handgrip portion and access the control surface. For example, as shown in FIG. 1, it is contemplated that the control surface can be positioned substantially directly above the handgrip portion when the device is held in an upright position. It is further contemplated that the control surface can be positioned substantially adjacent to an upper portion of the handgrip portion when the device is held in an upright position.

In another aspect, the system can comprise a light source. In this aspect, it is contemplated that the light source can comprise a conventional surgical-quality light source, such as, for example and without limitation, a fiber optic light source. In one aspect, when the endoscope comprises a light post, the passage of the light post can be configured to receive the light source using conventional means such that the light source is in communication with the lens assembly of the endoscope.

In yet another aspect, the system can comprise a transmission cable that is operatively coupled thereto and extends therebetween the light source and the centrifugal bore of the endoscope. In an exemplary aspect, when the proximal end portion of the endoscope defines an aperture, the transmission cable can be operatively coupled thereto and extend therebetween the light source and the aperture of the proximal end portion of the endoscope. In this aspect, the transmission cable can be configured to transmit light into the centrifugal bore of the endoscope substantially along the longitudinal axis of the endoscope. It is contemplated that the transmission cable can be coupled to the aperture of the proximal end portion of the endoscope using conventional means, including, for example and without limitation, conventional fiber optic ring couplers. It is further contemplated that the usage of the aperture defined in the proximal end portion of the endoscope can eliminate the need for a conventional light portal.

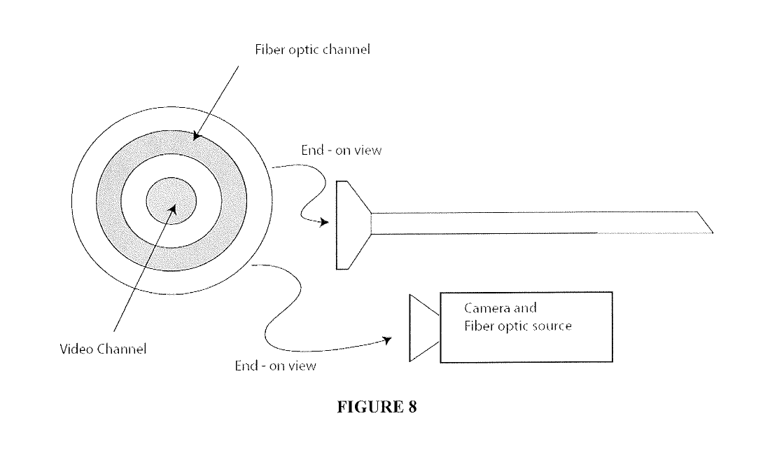

In an additional aspect, the device can comprise a camera operatively coupled to the video channel of the endoscope such that the camera is configured to capture an image produced by the lens assembly of the endoscope. Optionally, in this aspect, and with reference to FIG. 8, it is contemplated that the transmission cable can comprise a light guide and a video channel with the light guide of the transmission cable providing operative communication between the light source and the centrifugal bore of the endoscope and the video channel of the transmission cable providing operative communication between the camera and the centrifugal bore of the endoscope. Alternatively, in another optional aspect, the camera can be operatively coupled to the viewing portal of the endoscope (when present). Optionally, in exemplary aspects, the camera can be positioned therein the internal compartment of the housing. In another aspect, it is contemplated that the camera can be in operative communication with a display device such that the images received by the camera are displayed on the display device. In this aspect, it is contemplated that the display device can be any conventional display device, including, for example and without limitation, an external monitor screen. It is further contemplated that the camera can be placed in operative communication with the display device through any conventional electrical communication means, including, for example, wired and wireless communication means.

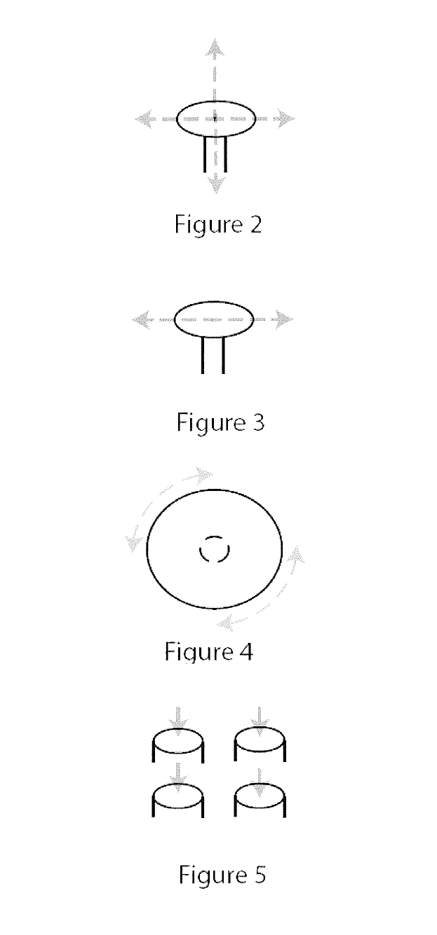

In a further aspect, the system can comprise a user input assembly configured to permit entry of user input controls. In this aspect, the user input controls can correspond to a selected rotational position of the endoscope. In additional aspects, it is contemplated that the user input controls can correspond to light activation controls corresponding to a desired activation or inactivation of a light source. It is contemplated that the user input assembly can be configured to permit one-handed, selective control of the rotation of the endoscope about the longitudinal axis of the endoscope. In an additional aspect, the user input assembly can optionally be positioned thereon the control surface of the housing. It is contemplated that the input assembly can comprise any conventional user interface, including, for example and without limitation, one or more of a joystick, a rotary encoder, a button, and the like. For example, in one aspect, as shown in FIG. 3, the user input assembly can comprise a joystick configured for movement along a single axis. In another aspect, as shown in FIG. 2, the user input assembly can comprise a joystick configured for movement along at least two axes. In still another aspect, as shown in FIG. 4, the user input assembly can comprise a rotary encoder. In yet another aspect, as shown in FIG. 5, the user input assembly can comprise a plurality of buttons. In this aspect, it is contemplated that the plurality of buttons can be in the form of a keypad or a keyboard.

In another aspect, the system can comprise control circuitry in operative communication with the user input assembly. In this aspect, the control circuitry can be configured to receive the user input controls entered into the user input assembly, including the user input controls corresponding to the selected rotational position of the endoscope and the light activation controls, as described herein. Optionally, the control circuitry can be positioned within the internal compartment of the housing. In exemplary aspects, the control circuitry can comprise a processor. In these aspects, it is contemplated that the processor can be operatively, electrically coupled thereto the user input assembly.

In another exemplary aspect, the processor can be a part of a microcontroller positioned therein the internal compartment of the housing. In this aspect, it is contemplated that the processor, and the microcontroller, can be placed in operative communication with a display device configured to display selected data and other predetermined outputs of the processor. It is contemplated that the display device can be any conventional display device, including, for example and without limitation, an external monitor screen. It is further contemplated that the processor, and microcontroller, can be placed in operative communication with the display device through any conventional electrical communication means, including, for example, wired and wireless communication means. In an alternative, exemplary aspect, the processor can be a part of a computer positioned external to the housing.

In still a further aspect, the system can comprise an actuator configured to controllably rotate the endoscope about the longitudinal axis of the endoscope in response to the received user input controls. In this aspect, it is contemplated that the actuator can be any conventional means for effecting rotation movement, including, for example and without limitation, an electric motor, a hydraulic pump, an air cylinder, a linear actuator, and the like. In exemplary aspects, the actuator can comprise a stepper motor. In one aspect, it is contemplated that the actuator can be operatively coupled to the proximal end portion of the endoscope such that selective activation of the actuator by the processor results in a corresponding rotation of the endoscope. It is contemplated that the actuator can be operatively coupled to the proximal end portion of the endoscope using any conventional electromechanical mechanism, including, for example and without limitation, reduction gearing, coupling brackets, and the like. In one exemplary aspect, the In another exemplary aspect, the actuator can comprise a servo motor, such as, for example and without limitation, an RC servo motor.

In an additional aspect, the system can comprise a position sensor. In this aspect, it is contemplated that the position sensor can comprise any conventional means for sensing the rotational position of an object, including, for example and without limitation, an optical encoder, a capacitive encoder, a synchro, a potentiometer, and various conventional transducers that are configured to measure the rotational position of an element relative to a predetermined reference position. In this aspect, the position sensor can be configured to produce a position signal indicative of the axial position of the endoscope relative to a reference position. It is contemplated that the position sensor can be positioned in operative communication with the control circuitry, including the processor, using conventional means.

In exemplary aspects, it is contemplated that the processor can be operatively coupled to the user input assembly, the actuator, and the position sensor. In one aspect, the processor can be configured to compare the rotational position of the endoscope with the selected rotational position entered into the user input assembly by a user. In this aspect, the processor can be further configured to determine a required axial rotation of the endoscope that is sufficient to position the endoscope in the selected rotational position to thereby achieve a desired angular position of the objective lens. It is contemplated that the processor can be still further configured to instruct the actuator to effect the required axial rotation of the endoscope. In further aspects, it is contemplated that, following positioning of the endoscope in the selected rotational position, the position sensor can be configured to periodically transmit an updated position signal to the processor for verification that the selected rotational position of the endoscope is maintained. In these aspects, the updated position signals can be used to determine what angular rotation, if any, is necessary, to return the endoscope to the desired angular position. After this determination is made, the processor can instruct the actuator to effect the appropriate axial rotation of the endoscope to return the endoscope to the desired angular position. Thus, it is contemplated that the processor, the actuator, the memory, and the position sensor cooperate to provide rotation correction to the endoscope to thereby obtain and maintain a desired rotational position.

In another aspect, it is contemplated that the light source can be in operative communication with the control circuitry. In an exemplary aspect, the light source can be in operative communication with the processor. In this aspect, it is contemplated that the light source can be configured to controllably generate light (or cease light generation) in response to one or more received light activation controls.

In a further aspect, the control circuitry can further comprise a memory coupled to the processor. As will be appreciated by one skilled in the art, the memory of the device can comprise software for using the user input controls and the position signal to determine the axial rotation of the endoscope that is required to achieve the axial position of the endoscope corresponding to the user input controls. In exemplary aspects, the memory can be part of the microcontroller positioned within the internal compartment of the housing. However, it is contemplated that the memory can alternatively be positioned external to the housing.

In a further aspect, the system can comprise a power source placed in electrical communication with the control circuitry. In this aspect, it is contemplated that the power source can be a conventional wireless power source, such as, for example and without limitation, a battery. Alternatively, it is contemplated that the power source can comprise a conventional AC power adapter.

In still another aspect, the system can comprise camera focusing means coupled to the camera. In this aspect, the camera focusing means can comprise any conventional mechanisms for focusing an image obtained by a camera, including, for example and without limitation, a conventional manual focus, an electromechanically augmented optical focus such as conventionally found in point-and-shoot commercial-quality cameras, and the like. It is contemplated that manual focus means can be externally mounted thereon the camera, whereas automatic focus means can comprise conventional digital processing techniques for analyzing an imaging and adjusting the focus as appropriate. In an exemplary aspect, the camera focusing means can comprise an electromechanical device for adjusting the focal length of the camera, with the electromechanical device being operatively coupled to the user input assembly via the control circuitry. In this aspect, it is contemplated that the user input control can be configured to receive a focal length control corresponding to a desired focal length for the camera, and the focal length control can be transmitted to the electromechanical device through the control circuitry.

In use, the disclosed devices and systems can be incorporated into methods for controlling endoscopic imaging. An exemplary method for controlling endoscopic imaging can comprise positioning the distal end portion of the endoscope in a desired location within the body of a subject. The method can further comprise receiving one or more user input controls through the input assembly to selectively control the rotation of the endoscope along the longitudinal axis of the endoscope. The method can further comprise transmitting the received user input controls to the processor.

In an exemplary aspect, the method can further comprise the step of instructing, through the processor, the actuator to cause rotation of the endoscope corresponding to the received user input controls.

In another exemplary aspect, when the device further comprises a position sensor, the method can further comprise, through the position sensor, transmitting to the processor a position signal indicative of the rotational position of the endoscope. In this aspect, the method can further comprise the step of determining, through the processor, the required axial rotation of the endoscope that is sufficient to position the endoscope in the selected rotational position corresponding to the received user input controls. The method can still further comprise, through the processor, instructing the actuator to effect the required axial rotation of the endoscope.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed hereinabove, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

What is claimed is:

1. An endoscopic imaging system comprising:
an endoscope having a longitudinal axis, a proximal end portion, and a distal end portion;
a user input assembly configured to permit entry of user input controls, the user input controls corresponding to a selected rotational position of the endoscope;
control circuitry in operative communication with the user input assembly, the control circuitry being configured to receive the user input controls;
an actuator in operative communication with the control circuitry, the actuator being configured to controllably rotate the endoscope about the longitudinal axis of the endoscope in response to the received user input controls; and
a position sensor in operative communication with the control circuitry, the position sensor being configured to produce a position signal indicative of the rotational position of the endoscope.

2. The endoscopic imaging system of claim 1, wherein the control circuitry is configured to compare the rotational position of the endoscope with the selected rotational position.

3. The endoscopic imaging system of claim 1, wherein the control circuitry is configured to determine a required axial rotation of the endoscope that is sufficient to position the endoscope in the selected rotational position, and wherein the control circuitry is further configured to instruct the actuator to effect the required axial rotation of the endoscope.

4. An endoscopic imaging system comprising:
a rigid endoscope defining a centrifugal bore and having a longitudinal axis, a proximal end portion, and a distal end portion, the endoscope comprising a lens assembly positioned therein the centrifugal bore, the lens assembly having an objective lens positioned proximate the distal end portion of the endoscope, the objective lens being angularly oriented relative to the longitudinal axis of the endoscope, the proximal end portion defining a viewing portal surrounding the longitudinal axis of the endoscope;
a housing having an internal compartment, the housing securely coupled to the proximal end portion of the endoscope;
a user input assembly configured to permit entry of user input controls, the user input controls corresponding to a selected rotational position of the endoscope;
control circuitry positioned within the internal compartment of the housing and in operative communication with the user input assembly, the control circuitry being configured to receive the user input controls;
an actuator positioned within the internal compartment of the housing and in operative communication with the control circuitry, the actuator being configured to controllably rotate the endoscope about the longitudinal axis of the endoscope in response to the received user input controls; and
a position sensor positioned within the internal compartment of the housing and in operative communication with the control circuitry, the position sensor being configured to produce a position signal indicative of the rotational position of the endoscope.

5. The endoscopic imaging system of claim 4, wherein the control circuitry comprises a processor, and wherein the processor is operatively coupled thereto the user input assembly, the actuator, and the position sensor.

6. The endoscopic imaging system of claim 4, wherein the actuator comprises an electric stepper motor operatively coupled to the proximal end portion of the endoscope.

7. The endoscopic imaging system of claim 5, wherein the processor is configured to compare the rotational position of the endoscope with the selected rotational position.

8. The endoscopic imaging system of claim 7, wherein the processor is configured to determine a required axial rotation of the endoscope that is sufficient to position the endoscope in the selected rotational position, and wherein the processor is further configured to instruct the actuator to effect the required axial rotation of the endoscope.

9. The endoscopic imaging system of claim 4, wherein the user input assembly comprises a joystick.

10. The endoscopic imaging system of claim 9, wherein the joystick is configured for selective movement along a single axis.

11. The endoscopic imaging system of claim 9, wherein the joystick is configured for selective movement along at least two axes.

12. The endoscopic imaging system of claim 4, wherein the user input assembly comprises a rotary encoder.

13. The endoscopic imaging system of claim 4, wherein the user input assembly comprises a plurality of buttons.

14. The endoscopic imaging system of claim 4, wherein the housing comprises a handgrip portion configured to conform to the shape of a user's hand.

15. The endoscopic imaging system of claim 4, wherein the objective lens of the endoscope is angularly oriented relative to the longitudinal axis of the endoscope by about 30° to about 70°.

16. The endoscopic imaging system of claim 4, wherein the processor is part of a microcontroller positioned therein the internal compartment of the housing.

17. The endoscopic imaging system of claim 4, wherein the user input assembly is positioned thereon a portion of the housing.

18. An endoscopic imaging system comprising:
a rigid endoscope defining a centrifugal bore and having a longitudinal axis, a proximal end portion, and a distal end portion, the endoscope comprising a lens assembly positioned therein the centrifugal bore, the lens assembly having an objective lens positioned proximate the distal end portion of the endoscope, the objective lens being angularly oriented relative to the longitudinal axis of the endoscope, the proximal end portion of the endoscope defining an aperture circumferentially surrounding the longitudinal axis of the endoscope, the endoscope being configured for rotation about its longitudinal axis;
a housing having an internal compartment, the housing securely coupled to the proximal end portion of the endoscope;
a light source;
a fiber optic light guide optically coupled thereto and extending between the light source and the aperture of the proximal end portion of the endoscope, the fiber optic light guide configured to transmit light into the centrifugal bore of the endoscope substantially along the longitudinal axis of the endoscope without mechanically limiting rotation of the endoscope;
a user input assembly configured to permit entry of user input controls, the user input controls corresponding to a selected rotational position of the endoscope;
control circuitry in operative communication with the user input assembly, the control circuitry being configured to receive the user input controls;
an actuator in operative communication with the control circuitry, the actuator being configured to controllably rotate the endoscope about the longitudinal axis of the endoscope in response to the received user input controls;
a position sensor in operative communication with the control circuitry, the position sensor being configured to produce a position signal indicative of the rotational position of the endoscope.

19. The endoscopic imaging system of claim 18, further comprising a video camera operatively coupled to the centrifugal bore of the endoscope such that the camera is configured to receive an image produced by the lens assembly.

20. The endoscopic imaging system of claim 19, further comprising a video channel, wherein the video channel of the transmission cable provides operative communication between the video camera and the centrifugal bore of the endoscope.

21. The endoscopic imaging system of claim 19, further comprising camera focusing means operatively coupled thereto the camera.

22. The endoscopic imaging system of claim 18, wherein the user input assembly is configured to permit entry of a light activation control, wherein the control circuitry is configured to receive the light activation control, and wherein the light source is in operative communication with the control circuitry and configured to controllably generate light in response to the received light activation control.

23. An endoscopic imaging process comprising:
providing an endoscope having a longitudinal axis, a proximal end portion, and a distal end portion;
receiving input comprising user input controls, the user input controls corresponding to a selected rotational position of the endoscope;
controllably rotating the endoscope about the longitudinal axis of the endoscope in response to the received user input controls; and
producing a position signal indicative of the rotational position of the endoscope.

24. The endoscopic imaging process of claim 23, further comprising the step of comparing the rotational position of the endoscope with the selected rotational position.

25. The endoscopic imaging process of claim 23, further comprising the steps of determining a required axial rotation of the endoscope that is sufficient to position the endoscope in the selected rotational position, and effecting the required axial rotation of the endoscope.
